(19) United States
(12) Patent Application Publication
(10) Pub. No.: US 2005/0272972 A1
(43) Pub. Date: Dec. 8, 2005

(54) Method, system and device for suction biopsy

(76) Inventor: Gavriel J. Iddan, Haifa (IL)

Correspondence Address: PEARL COHEN ZEDEK, LLP, 10 ROCKEFELLER PLAZA, SUITE 1001, NEW YORK, NY, 10020, US

(21) Appl. No.: 11/146,017

(22) Filed: Jun. 7, 2005

Related U.S. Application Data
(60) Provisional application No. 60/577,203, filed on Jun. 7, 2004.

(51) Int. Cl. A61B 1/00

(52) U.S. Cl. 600/102

(57) ABSTRACT

A device, system and method for suction biopsy. An autonomous in-vivo device may include a suction chamber to store a sample; a plunger movable into an inner portion of said suction chamber, an imager to acquire in-vivo an image; and a transmitter to transmit said image.

### PRIOR APPLICATION DATA

This application claims priority and benefit from U.S. Provisional Patent Application No. 60/577,203, entitled “Method, System and Device for Suction Biopsy”, filed on Jun. 7, 2004, and incorporated herein by reference in its entirety.

### FIELD OF THE INVENTION

The present invention relates to the field of biopsies of in-vivo areas, for example, endo-luminal areas.

### BACKGROUND OF THE INVENTION

Biopsies or the taking of samples of endo-luminal tissue are a widely used diagnostic tool for diseases such as, Gastro-Intestinal (GI) tract diseases, for example Helicobacter pylori, celiac disease and Crohn's disease. The diagnosis of other diseases may likewise heavily rely on biopsies of tissues from in-vivo While surgery or endoscopy may sometimes be used to collect biopsy samples, such procedures can be uncomfortable for the patient, as well as time consuming and expensive to administer.

### SUMMARY OF THE INVENTION

Various embodiments of the invention provide, for example, devices, systems and methods of suction biopsy, e.g., in-vivo suction biopsy.

In some embodiments, for example, an autonomous in-vivo device may include a suction chamber to store a sample, a plunger movable into an inner portion of the suction chamber, and optionally a blade to the sample from a lumen wall. The autonomous in-vivo device may optionally include an imager to acquire in-vivo an image (e.g., of the sample, the suction chamber, a body lumen, or some of the above), and a transmitter to transmit the image.

In some embodiments, for example, the in-vivo device may include a coil to move the plunger towards the inner portion of the suction chamber. The coil may be activated, for example, by an electric current.

In some embodiments, for example, the in-vivo device may include a latch to hold the plunger at the inner portion of the suction chamber.

In some embodiments, for example, the blade may be activated by a movement of the plunger into the inner portion of the suction chamber.

In some embodiments, for example, the in-vivo device may include a spring to snap shut the blade over the suction chamber upon movement of the plunger into the inner portion of the suction chamber.

In some embodiments, for example, the blade may be held in place, prior to its activation, by the plunger.

In some embodiments, for example, at least a circumferential edge of the plunger includes a lubricant.

In some embodiments, for example, at least a portion of the plunger may be surrounded by a sealant.

In some embodiments, for example, a movement of the plunger may be triggered by an external command.

In some embodiments, for example, the in-vivo device may include an in-vivo sensor to trigger a movement of the plunger.

In some embodiments, for example, a movement of the plunger may be triggered based on a location of the in-vivo device.

In some embodiments, for example, the in-vivo device may include a plurality of suction chambers to collect a plurality of samples, respectively.

In some embodiments, for example, the plurality of suction chambers may be positioned at a plurality of sides of the in-vivo device, respectively.

In some embodiments, for example, a first of the suction chambers may collect a first sample at a first time, and a second of the suction chambers may collect a second sample at a second, different time.

In some embodiments, for example, at least two of the suction chambers are to collect the samples substantially simultaneously.

In some embodiments, for example, the in-vivo device may include a rotatable wheel having at least two blades to cut the samples.

In some embodiments, for example, the in-vivo device may include an in-vivo sensor to sense a property of the sample stored in the suction chamber, e.g., temperature, pH, pressure, bacteria, an optical quality, an optical characteristic, an image, or the like.

In some embodiments, for example, the in-vivo device may include an in-vivo camera to acquire an in-vivo image of a body lumen.

In some embodiments, for example, the in-vivo device may include an in-vivo imager to acquire an image of the sample stored in the suction chamber.

In some embodiments, for example, the in-vivo device may include a transmitter to transmit the image.

In some embodiments, a system may include, for example, an in-vivo device including at least a suction chamber to store a sample, a plunger movable into an inner portion of the suction chamber, and a blade to cut or slice the sample; and a receiver to receive data transmitted from the in-vivo device.

In some embodiments of the system, for example, the in-vivo device may include an in-vivo sensor to sense a property of the sample stored in the suction chamber; and a transmitter to transmit the sensed data.

In some embodiments of the system, for example, the in-vivo device may include an in-vivo imager to acquire an image of the sample stored in the suction chamber; and a transmitter to transmit the image data.

In some embodiments of the system, for example, the in-vivo device may include an in-vivo camera to acquire an image of a body lumen; and a transmitter to transmit the image data.

In some embodiments, a method may include, for example, creating suction in a suction chamber of an in-vivo device; drawing in a body tissue into the suction chamber using the suction; and slicing a sample of the tissue.

In some embodiments, the method may include, for example, acquiring an in-vivo image of the sample, sensing in-vivo a property of the sample, and/or analyzing in-vivo the sample.

Some embodiments may include, for example, an in-vivo device which may be autonomous and/or may include a swallowable capsule.

Embodiments of the invention may allow various other benefits, and may be used in conjunction with various other applications.

### BRIEF DESCRIPTION OF THE DRAWINGS

The subject matter regarded as the invention is particularly pointed out and distinctly claimed in the concluding portion of the specification The invention, however, both as to organization and method of operation, together with objects, features and advantages thereof, may best be understood by reference to the following detailed description when read with the accompanied drawings in which:

FIG. 1 is a schematic diagram of a suction chamber in accordance with an embodiment of the invention;

FIG. 2 is a schematic diagram of a suction chamber with a plunger that has been lowered in accordance with an embodiment of the invention;

FIG. 3A is a side view of an autonomous in-vivo device including a suction chamber and a blade cover in accordance with an embodiment of the invention;

FIG. 3B is a cut-away view of an autonomous in-vivo device including a plurality of suction chambers around a circumference of the in-vivo device in accordance with an embodiment of the invention;

FIG. 4A is a side view of a rotating turret with blades that may be used to slice off a sample, in accordance with an embodiment of the invention;

FIG. 4B is a front view of a rotating turret with blades that may be used to slice off a sample, in accordance with an embodiment of the invention;

FIG. 4C is a view of an autonomous in-vivo device with a rotating turret with blades that may be used to slice off a sample, in accordance with an embodiment of the invention:

FIG. 5 is a flow chart of a method of sampling in-accordance with an embodiment of the invention;

FIG. 6 is a view of an autonomous in-vivo device with a sample having been sliced into a suction chamber in accordance with an embodiment of the invention; and

FIG. 7 is a schematic illustration of an in-vivo system in accordance with an embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

Various examples are given throughout this description. These are merely descriptions of specific embodiments of the invention, but the scope of the invention is not limited to the examples given. Features described with respect to one embodiment may be included in other embodiments though not described therein. Aspects of the various embodiments disclosed herein are combinable with the other embodiments disclosed herein.

It should be noted that although a portion of the discussion may relate to in-vivo imaging devices, systems, and methods, the present invention is not limited in this regard, and embodiments of the present invention may be used in conjunction with various other in-vivo sensing devices, systems, and methods. For example, some embodiments of the invention may be used, for example, in conjunction with in-vivo sensing of pH, in-vivo sensing of temperature, in-vivo sensing of pressure, in-vivo sensing of electrical impedance, in-vivo detection of a substance or a material, in-vivo detection of a medical condition or a pathology, in-vivo acquisition or analysis of data, and/or various other in-vivo sensing devices, systems, and methods. Some embodiments of the invention may be used not necessarily in the context of in-vivo imaging or in-vivo sensing.

Some embodiments of the present invention are directed to a typically swallowable in-vivo sensing device, e.g., a typically swallowable in-vivo imaging device. Devices according to embodiments of the present invention may be similar to embodiments described in U.S. patent application Ser. No. 09/800,470, entitled “Device And System For In-vivo Imaging”, filed on 8 Mar., 2001, published on Nov. 1, 2001 as U.S. patent application Publication No. 2001/0035902, and/or in U.S. Pat. No. 5,604,531 to Iddan et al., entitled “In-Vivo Video Camera System”, and/or in International Application number WO 02/054932 entitled “System and Method for Wide Field Imaging of Body Lumens” published on Jul. 18, 2002, all of which are hereby incorporated by reference. An external receiving unit and processor, such as in a work station, such as those described in the above publications could be suitable for use with embodiments of the present invention. Devices and systems as described herein may have other configurations and/or other sets of components. For example, the present invention may be practiced using an endoscope, needle, stent, catheter, etc.

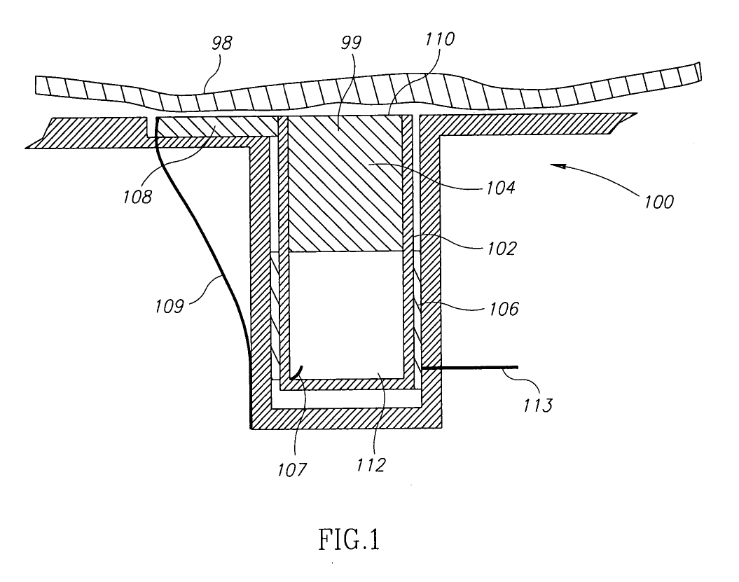

Reference is made to FIG. 1, a schematic drawing of a suction or sampling chamber 100 in accordance with an embodiment of the invention Suction or sampling chamber 100 may include, for example, a bucket 102, a plunger 104, a coil 106, and a cutting element or blade 108, e.g., a spring loaded blade, a wire cutting element, or other suitable cutting element.

In some embodiments, suction chamber 100 may be included in, or may be part of, an autonomous in-vivo device, such as a capsule that may be inserted or ingested into an in-vivo area. Suction chamber 100 may be included in other in-vivo devices, such as endoscopes. Other items may be included in the suction chamber 100 and other configurations of elements are possible.

In some embodiments, suction chamber 100 may be cylindrically shaped with a diameter of from 0.5 mm to 1.5 mm. Other sizes and shapes are possible. Suction chamber 100 may, in one embodiment, have a depth of from 1 mm to 1.5 mm. Other sizes and dimensions may be used, and suction chamber 100 may be configured in different shapes, for example, square, oval, ellipse or other shapes.

In some embodiments, the walls of the suction chamber 100 may be constructed of plastic, rubber, inert metallic alloys, reactive materials or other suitable materials. In some embodiments, the walls of the suction chamber 100 may be made of a suitable material that establishes an air-tight seal with plunger 104.

In some embodiments, plunger 104 may be constructed of a metallic or magnetic substance, which is responsive to, or attracted by, an electromagnetic force. In some embodiments, plunger 104 may be slideably or moveably placed within suction chamber 100, so that plunger 104 may smoothly and with force be lowered or drawn into the bottom or lower portion 112 of suction chamber 100. In some embodiments, plunger 104 may create an air tight seal against the sides of suction chamber 100, so that as plunger 104 is lowered into the lower portion 112 of suction chamber 100, a vacuum, negative pressure differential, or suction force is created in the space between the upper portion 99 of plunger 104 and outside opening 110.

In some embodiments, the circumferential dimensions of plunger 104 may be approximately equal to (or slightly smaller than) the inside circumferential dimensions of suction chamber 100. In some embodiments, a sealant, for example, a rubber or elastic gasket, may surround or wrap plunger 104, e.g., to create an air tight seal between the edge of plunger 104 and the inside of suction chamber 100. In some embodiments, a lubricant, for example, an inert lubricant, may be applied (e.g., by coating, covering, attachment, or the like) to the circumferential edge of plunger 104, e.g., to enhance the seal between plunger 104 and the inside of suction chamber 100.

In some embodiments, plunger 104 may have a thickness of as little as 0.1 mm or thinner, when plunger 104 is, for example, fashioned of a fairly rigid material. Other thicknesses and configurations may be used. The thickness of plunger 104 may preferably leave sufficient space in suction chamber 100 for the collection of a sample 98 when plunger 104 is drawn into the bottom or lower portion 112 of suction chamber 100.

It is noted that when used herein, “upper” and “lower” are relative terms used for description only; in use the chamber 100 may be oriented any number of ways.

Blade 108 may be formed of a metallic, plastic or other suitable material. Blade 108 may be slideably situated contiguous to outside opening 110. In some embodiments, blade 108 may slide within a groove or track along an outside shell of the in-vivo device. A side of blade 108 may be held by, or may be in contact with, a spring 109 which may exert a sliding force against blade 108, so that blade 108 is forced in a direction that partially or completely closes outside opening 110. Another side of blade 108 may be sharpened to a cutting edge capable of cutting or slicing a sample 98 of, for example, endo-luminal tissue or other matter from the lumen wall, that may be sucked into suction chamber 100.

Suction chamber 100 may be brought into contact with, for example, an endo-luminal tissue or another area from which sample 98 of cells or a biopsy is desired to be taken. For example, suction chamber 100 may be included in an autonomous in-vivo device or capsule that may be inserted or ingested into, for example, a GI tract For example, in one embodiment, suction chamber 100 may have a distal end that terminates along the shell of the in-vivo device or capsule, and the shell may be brought into contact with an endo-luminal wall.

In operation, once an outside opening 110 of suction chamber 100 is brought into contact with an area from which a sample is desired to be taken, coil 106 may be activated. Activation of coil 106 may be by way of application of a current or charge from, for example, wires 113 to coil 106, so that an electromagnetic force is created around coil 106 and the lower part of suction chamber 100.

Upon the activation of coil 106, an electromagnetic force may forcibly attract plunger 104 towards the lower portion 112 of suction chamber 100. The passage of plunger 104 from the upper portion of suction chamber 100 near the outside opening 110 to the lower portion 112 of suction chamber 100 may, in some embodiments, create a negative pressure differential or suction in the space between outside opening 110 and the top of plunger 104. Such suction may be sufficient to draw into suction chamber 100 a portion or sample 98 of an endo-luminal tissue from the endo-luminal wall with which outside opening 110 may be in contact. In some embodiments, coil 106 may be deactivated once plunger 104 has been drawn into the lower portion 112 of suction chamber 100. In some embodiments, plunger 104 may be held by a latch 107 or other holding device that may secure it in place once it has been lowered into the lower portion 112 of suction chamber 100.

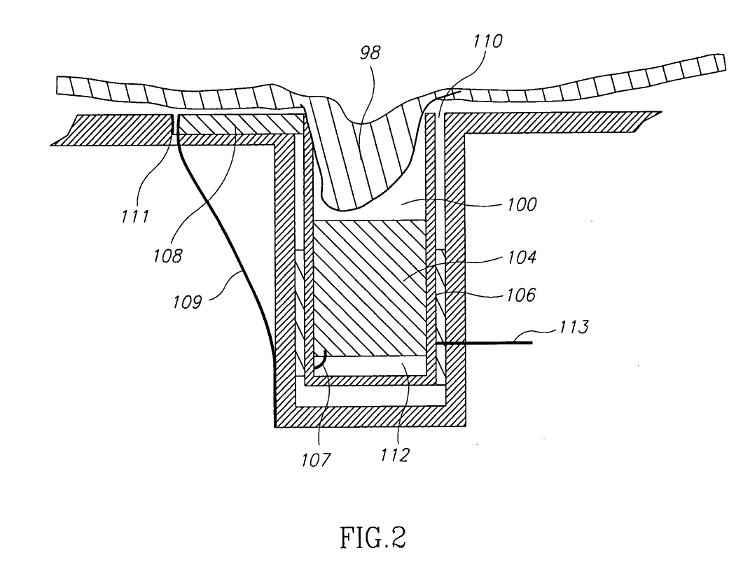

Reference is made to FIG. 2, a schematic depiction of a suction chamber 100 with a plunger that has been lowered in accordance with an embodiment of the invention. In some embodiments, blade 108 may be triggered to slide or move across outside opening 110 in synchronization with, or possibly a brief time afterwards, the exertion of a negative pressure differential or suction on outside opening 110. In some embodiments, the trigger that moves or activates blade 108 may be the lowering of plunger 104 into the lower portion 112 of suction chamber 100. In some embodiments, blade 108 may be held in an open position by plunger 104, and may be released when plunger 104 moves towards lower portion 112 or reaches a terminal position in lower portion 112.

In some embodiments, spring 109 may forcibly push blade 108 across outside opening 110, and outside opening 110 may be partially or entirely covered once blade 108 snaps shut across outside opening 110. In some embodiments, spring 109 may be held in an open position by a magnet, latch or other mechanism 111 until spring 109 is triggered to snap forward. Other mechanisms of moving blade 108 may be used, such as an electromagnetic force, a motor, a mechanical configuration, or the like.

In some embodiments, once blade 108 closes, the sample 98 that was cut by the closing blade 108 may be held in the space of suction chamber 100 between the lowered plunger 104 and the closed blade 108. The sample 98 may be retrieved for analysis or inspection once the in-vivo device, in which the suction chamber 100 is included, is expelled or removed from the body. In some embodiments, analysis of the sample may be performed in the in-vivo device, e.g. using a biopsy testing kit, a sensor, a sensing unit, an imager, an imaging unit, a processor, an analyzing unit, or the like.

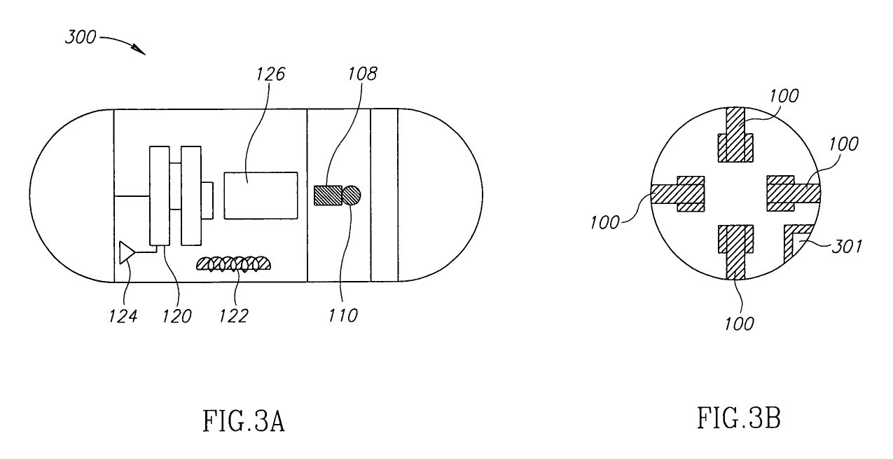

Reference is made to FIG. 3A, a side view of an autonomous in-vivo device including a suction chamber and a blade cover in accordance with an embodiment of the invention. Before blade 108 is released, blade 108 may be held next to outer opening 110. Once outside opening 110 is brought into contact with an endo-luminal wall or tissue, blade 108 may be released and snap shut over the outside opening 110. In some embodiments, device 300 may include components such as, for example, a power source 120, e.g., a battery; a transmitter 122 or transceiver, typically operating wirelessly via Radio Frequency (RF), microwave, or other the like; an antenna 124 or antenna array, e.g., to transmit images or other data; control circuitry 126 and/or other control mechanisms, or other suitable components.

In one embodiment, transmitter 122 may include control capability for, for example, controlling the various operations of device 300, although control capability or one or more aspects of control may be included in a separate component. Transmitter 122 may include or be a control circuit such as, for example, an Application Specific Integrated Circuit), but may be of other constructions; for example, transmitter 122 may be a processor executing instructions. Device 300 may include a processing unit separate from transmitter 122 that may, for example, contain or process instructions.

Device 300 typically may be or may include an autonomous in-vivo device, e.g., a swallowable capsule, but device 300 may have other shapes and need not be swallowable and/or autonomous. For example, device 300 may be a capsule or other unit where all the components are substantially contained within a container or shell or housing, and where device 300 does not require any wires or cables to, for example, receive power or transmit information. Device 300 may communicate with an external receiving and display system to provide display of data, control, or other functions. Power may be provided to device 300 by an internal battery or, for example, a wireless receiving system. Other embodiments may have other configurations and capabilities. For example, components may be distributed over multiple sites or units. Control information may be received from an external source

Reference is made to FIG. 3B, a cut-away view of an autonomous in-vivo device including a plurality of suction chambers around a circumference of the in-vivo device in accordance with an embodiment of the invention. In some embodiments, multiple suction chambers 100 may be situated around a circumference of an in-vivo device. Each of the suction chambers 100 may be capable of collecting and holding a sample, e.g., of tissue. The placement and/or positioning of suction chambers 100 around the circumference of device 300 may allow multiple samples 98 to be taken when, for example, an endo-luminal tissue is in contact with different parts, sides or areas of the in-vivo device. For example, if a bottom portion of the in-vivo device is in contact with an area to be sampled, a suction chamber 100 at the bottom portion may be activated to collect sample 98. At another time, a different area of the in-vivo device near another suction chamber 100 may be in contact with an endo-luminal area, and the suction chamber 100 that is then in contact may be activated to collect another sample. In some embodiments, an orientation, position or location mechanism 301 may be included in the in-vivo device to indicate, for example, the position or orientation of the in vivo device at the time that a sample was collected, or the location of the in-vivo device in a body lumen at the time that the sample was taken.

In some embodiments, device 300 may include an imaging unit or imager that may capture images of the body lumen, of any component(s) of device 300, of the suction chamber 100, of the blade 108 or of outside opening 110. In some embodiments, a user, for example, an external operator, may view the acquired in-vivo images of the body lumen or of any of, for example, device 300, suction chamber 100, blade 108 or outside opening 100, and may time or activate the slicing of a tissue or other sample 98 to coincide with a particular event, location or position of device 300.

For example, an external user may view images of the body lumen wall, and may detect a pathology or a suspected area on the body lumen wall; based on the detection, a decision may be made to activate the suction chamber 100 and/or blade 108. In some embodiments, an external user may receive a reading from, for example, a location sensor or mechanism 301 in device 300, and may signal the blade 108 to cut a slice of sample 98 based on a reading from such location sensor. Other kinds of sensors may be used, and other indications may be used as a trigger for the activation of blade 108 and/or the collection of sample 98 into one or more suction chamber(s) 100.

According to some embodiments, the in-vivo device may include a suction chamber 100 but need not include an image sensor, an imager, or other type of sensor.

In one embodiment, a sensor included in device 300 may automatically trigger the activation of suction chamber 100 to collect a sample of a tissue. For example, an image sensor, a pH sensor, a temperature sensor, a blood monitor, a pressure sensor, or other sensor may collect a reading that may, for example, be processed or analyzed by a processor; based on the analysis, the processor may trigger one or more suction chambers 100 to collect one or more samples 98 of tissue in a particular area where a reading was taken.

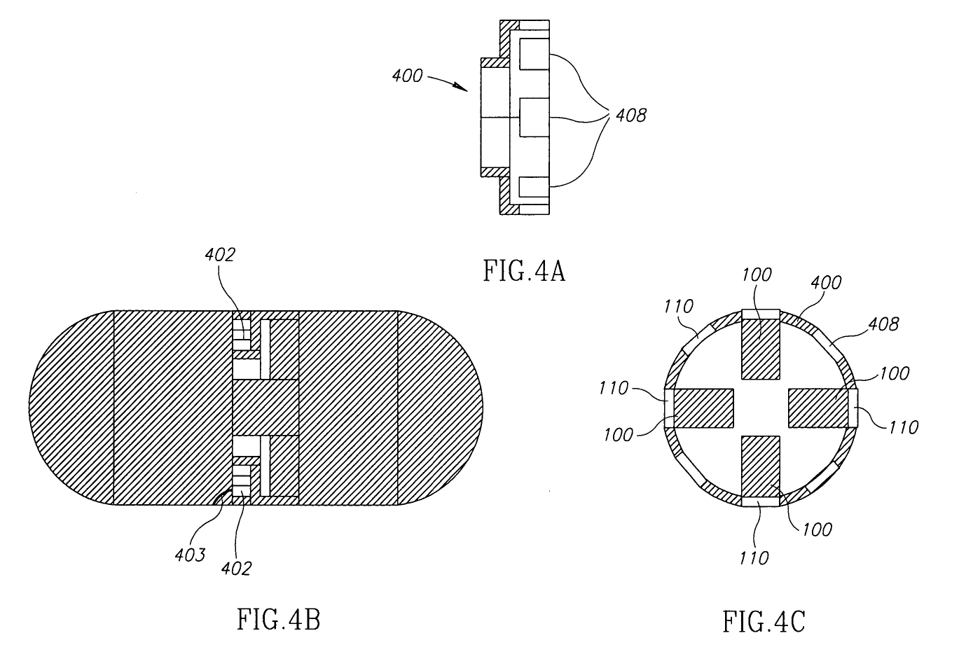

Reference is made to FIG. 4A, a side view of a rotating blade that may be used to slice off or cut off one or more samples, in accordance with an embodiment of the invention; to FIG. 4B, a front view of a rotating turret with blades that may be used to slice off or cut off one or more samples, in accordance with an embodiment of the invention; and to FIG. 4C, a view of an autonomous in-vivo device with a rotating turret with blades that may be used to slice off or cut off a sample, in accordance with an embodiment of the invention.

In some embodiments, there may be situated on a rotating turret 400 (e.g., a holder, a wheel, a mounting, a support, or the like) one or more blades 408 that may be driven forward by, for example, a spring 402 in increments of, for example, a quarter turn. Other increments may be used. Other methods of driving turret 400, such as a motor, an electromagnetic force, etc., may be used. When spring 402 is released by, for example, a catch, latch, switch, electromagnetic device or other component 403, rotating turret 400 may be driven forward in, for example, a circular motion When the turret 400 spins or is otherwise driven forward, one or more blades 408 may cover over an outside opening 110 of one or more suction chambers 100, and may slice or cut a sample 98 that was sucked into one or more of the suction chambers 100. In some embodiments, all of the blades 408 may shut over all of the suction chambers 100 when rotating turret 400 is spun forward. In some embodiments there may be more than one rotating turret 400 and only a single blade 408 may move over a single suction chamber 100. Other combinations are possible.

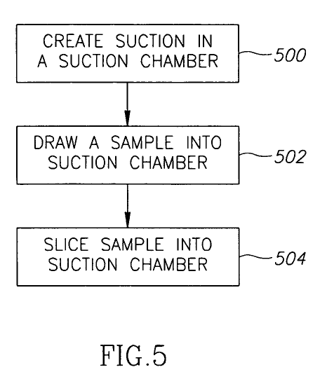

Reference is made to FIG. 5, a flow chart of a method of sampling in accordance with an embodiment of the invention. In block 500, suction or negative pressure differential may be created at the upper portion of a suction chamber. In some embodiments, such suction may be created by, for example, a plunger that is drawn into the lower portion of the suction chamber. In some embodiments, an internal or externally generated trigger or signal may, for example, cause plunger to be drawn into the lower portion of suction chamber.

In block 502, a sample of tissue, endo-luminal wall, GI tract liquids, or other material or substance may be drawn into the suction chamber, e.g., by the suction or negative pressure differential. In block 504, a slice of a tissue or other sample may be cut from, for example, an endo-luminal wall and stored in the suction chamber.

Other suitable operations may be performed; for example, the suction chamber may be closed or covered, e.g., by the blade or another component; the sample may be analyzed in-vivo, sensed in-vivo, or imaged in-vivo; or other suitable operations may be performed.

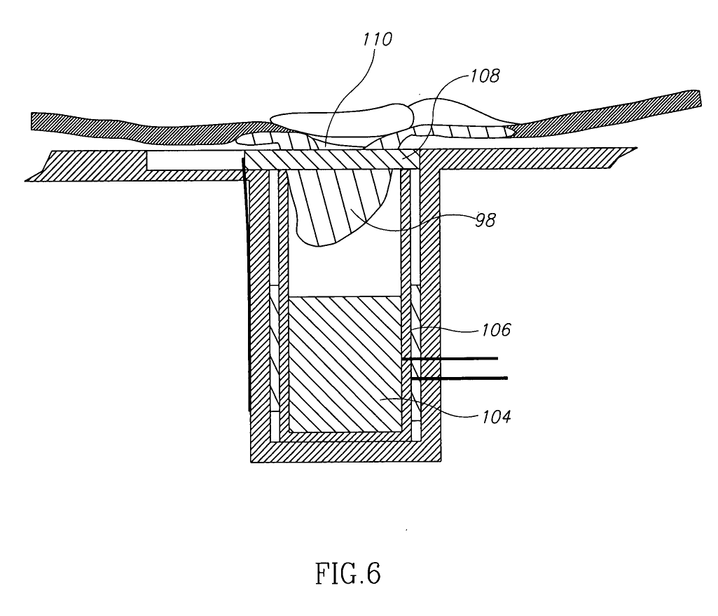

FIG. 6 is a view of an autonomous in-vivo device with a sample having been sliced or cut into a suction chamber 100 in accordance with an embodiment of the invention. In some embodiments, blade 108 may snap forward covering outside opening 110 and slicing off a sample 98 to be retained in suction chamber 100. Coil 106 may, in some embodiments, be deactivated and plunger 104 may remain at the bottom (or lower portion) of suction chamber 100.

In some embodiments, a sample or tissue that was stored in the suction chamber 100 may be retrieved, for example, when the in-vivo device in which it was stored is excreted or otherwise removed from the body. In some embodiments, the suction chamber 100 may contain a preservative to preserve the freshness (or other properties) of the stored sample until it is retrieved and/or analyzed In some embodiments, the suction chamber 100 may include an imager or other analysis device by which a sample may be viewed or tested in accordance with various physiological parameters, such as, for example, pH, bacterial content, temperature, pressure, presence of blood, or the like. Other methods of analysis are possible.

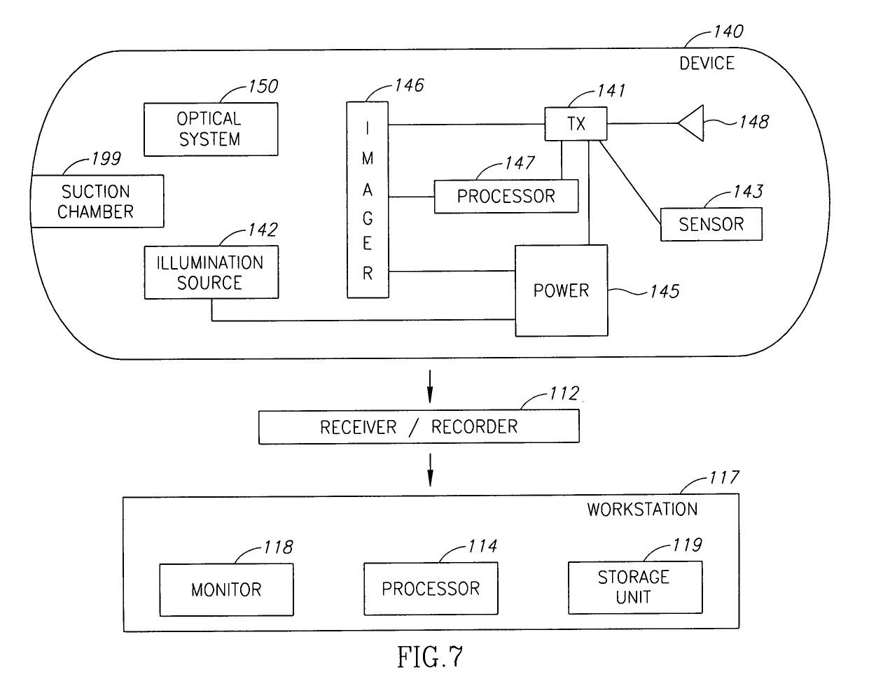

FIG. 7 shows a schematic illustration of an in-vivo system in accordance with some embodiments of the present invention. One or more components of the system may be used in conjunction with, may be operatively associated with, or may be included in, the devices and/or components of FIGS. 1-6, or other in-vivo devices in accordance with embodiments of the invention.

In one embodiment, the system may include a device 140 having a sensor, e.g., an imager 146, one or more illumination sources 142, a power source 145, and a transmitter 141. In some embodiments, device 140 may be implemented using a swallowable capsule, but other sorts of devices or suitable implementations may be used. Outside a patient's body may be, for example, an external receiver/recorder 112 (including, or operatively associated with, for example, an antenna or an antenna array), a storage unit 119, a processor 114, and a monitor 118. In one embodiment, for example, processor 114, storage unit 119 and/or monitor 118 may be implemented as a workstation 117, e.g., a computer or a computing platform.

Transmitter 141 may operate using radio waves; but in some embodiments, such as those where device 140 is or is included within an endoscope, transmitter 141 may transmit/receive data via, for example, wire, optical fiber and/or other suitable methods. Other known wireless methods of transmission may be used. Transmitter 141 may include, for example, a transmitter module or sub-unit and a receiver module or sub-unit, or an integrated transceiver or transmitter-receiver.

Device 140 typically may be or may include an autonomous swallowable capsule, but device 140 may have other shapes and need not be swallowable or autonomous. Embodiments of device 140 are typically autonomous, and are typically self-contained. For example, device 140 may be a capsule or other unit where all the components are substantially contained within a container or shell, and where device 140 does not require any wires or cables to, for example, receive power or transmit information. In one embodiment, device 140 may be autonomous and non-remote-controllable; in another embodiment, device 140 may be partially or entirely remote-controllable.

In some embodiments, device 140 may communicate with an external receiving and display system (e.g., workstation 117 or monitor 118) to provide display of data, control, or other functions. For example, power may be provided to device 140 using an internal battery, an internal power source, or a wireless system able to receive power. Other embodiments may have other configurations and capabilities. For example, components may be distributed over multiple sites or units, and control information or other information may be received from an external source.

In one embodiment, device 140 may include an in-vivo video camera, for example, imager 146, which may capture and transmit images of, for example, the GI tract while device 140 passes through the GI lumen. Other lumens and/or body cavities may be imaged and/or sensed by device 140. In some embodiments, imager 146 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In one embodiment, imager 146 in device 140 may be operationally connected to transmitter 141. Transmitter 141 may transmit images to, for example, external transceiver or receiver/recorder 112 (e.g., through one or more antennas), which may send the data to processor 114 and/or to storage unit 119. Transmitter 141 may also include control capability, although control capability may be included in a separate component, e.g., processor 147. Transmitter 141 may include any suitable transmitter able to transmit image data, other sensed data, and/or other data (e.g., control data) to a receiving device. Transmitter 141 may also be capable of receiving signals/commands, for example from an external transceiver. For example, in one embodiment, transmitter 141 may include an ultra low power Radio Frequency (RF) high bandwidth transmitter, possibly provided in Chip Scale Package (CSP).

In some embodiment, transmitter 141 may transmit/receive via antenna 148. Transmitter 141 and/or another unit in device 140, e.g., a controller or processor 147, may include control capability, for example, one or more control modules, processing module, circuitry and/or functionality for controlling device 140, for controlling the operational mode or settings of device 140, and/or for performing control operations or processing operations within device 140. According to some embodiments, transmitter 141 may include a receiver which may receive signals (e.g., from outside the patient's body), for example, through antenna 148 or through a different antenna or receiving element. According to some embodiments, signals or data may be received by a separate receiving device in device 140.

Power source 145 may include one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to in-vivo device 140.

Optionally, in one embodiment, transmitter 141 may include a processing unit or processor or controller, for example, to process signals and/or data generated by imager 146. In another embodiment, the processing unit may be implemented using a separate component within device 140, e.g., controller or processor 147, or may be implemented as an integral part of imager 146, transmitter 141, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In one embodiment, for example, the processing unit or controller may be embedded in or integrated with transmitter 141, and may be implemented, for example, using an ASIC.

In some embodiments, device 140 may include one or more illumination sources 142, for example one or more Light Emitting Diodes (LEDs), “white LEDs”, or other suitable light sources. Illumination sources 142 may, for example, illuminate a body lumen or cavity being imaged and/or sensed. An optional optical system 150, including, for example, one or more optical elements, such as one or more lenses or composite lens assemblies, one or more suitable optical filters, or any other suitable optical elements, may optionally be included in device 140 and may aid in focusing reflected light onto imager 146, focusing illuminated light, and/or performing other light processing operations.

Data processor 114 may analyze the data received via external receiver/recorder 112 from device 140, and may be in communication with storage unit 119, e.g., transferring frame data to and from storage unit 119. Data processor 114 may provide the analyzed data to monitor 118, where a user (e g., a physician) may view or otherwise use the data. In one embodiment, data processor 114 may be configured for real time processing and/or for post processing to be performed and/or viewed at a later time. In the case that control capability (e.g., delay, timing, etc) is external to device 140, a suitable external device (such as, for example, data processor 114 or external receiver/recorder 112 having a transmitter or transceiver) may transmit one or more control signals to device 140.

Monitor 118 may include, for example, one or more screens, monitors, or suitable display units. Monitor 118, for example, may display one or more images or a stream of images captured and/or transmitted by device 140, e.g., images of the GI tract or of other imaged body lumen or cavity. Additionally or alternatively, monitor 118 may display, for example, control data, location or position data (e.g., data describing or indicating the location or the relative location of device 140), orientation data, and various other suitable data. In one embodiment, for example, both an image and its position (e.g., relative to the body lumen being imaged) or location may be presented using monitor 118 and/or may be stored using storage unit 119. Other systems and methods of storing and/or displaying collected image data and/or other data may be used.

Typically, device 140 may transmit image information in discrete portions. Each portion may typically correspond to an image or a frame; other suitable transmission methods may be used. For example, in some embodiments, device 140 may capture and/or acquire an image once every half second, and may transmit the image data to external receiver/recorder 112. Other constant and/or variable capture rates and/or transmission rates may be used.

Typically, the image data recorded and transmitted may include digital color image data; in alternate embodiments, other image formats (e.g., black and white image data) may be used. In one embodiment, each frame of image data may include 256 rows, each row may include 256 pixels, and each pixel may include data for color and brightness according to known methods. For example, a Bayer color filter may be applied. Other suitable data formats may be used, and other suitable numbers or types of rows, columns, arrays, pixels, sub-pixels, boxes, super-pixels and/or colors may be used.

Optionally, device 140 may include one or more sensors 143, instead of or in addition to a sensor such as imager 146. Sensor 143 may, for example, sense, detect, determine and/or measure one or more values of properties or characteristics of the surrounding of device 140. For example, sensor 143 may include a pH sensor, a temperature sensor, an electrical conductivity sensor, a pressure sensor, or any other known suitable in-vivo sensor. According to some embodiments, a sensor such as sensor 143 may be used to analyze (e.g., in-vivo) a sample in one or more of the suction chambers. For example, the pH of a sample may be sensed by a sensor on board the in-vivo device, and information from the sensor may be transmitted outside the body, for example, by transmitter 141 or by another transmitter receiving input from the sensor. In another embodiment, an in-vivo image sensor may be used to obtain color data (e.g., images) of a sample in a suction chamber. According to some embodiments, an imager on board the in-vivo device may image the suction chamber (and possibly a sample inside the suction chamber), and may transmit image data of the sample to an external receiver. In some embodiments, a body lumen and a sample may be imaged possibly simultaneously by the same imager, or separately using two imagers.

Device 140 may further include one or more components or mechanisms of any of FIGS. 1-6, and/or of other in-vivo devices in accordance with embodiments of the invention. Such components may include, for example, one or more suction chambers (e.g., a suction chamber 199), blades, plungers, covers, latches, springs, coils, turret, rotating parts, moving parts, or the like.

In some embodiments, in-vivo sensor 143 (or other suitable sensor) may sense or measure a property or characteristic of a content (e.g., a sample or substance) stored in suction chamber 199, for example, temperature, pH, pressure, optical quality, optical characteristic, color, brightness, hue, saturation, image, colorimetric characteristic, spectral characteristic, or the like.

Various aspects of the various embodiments disclosed herein are combinable with the other embodiments disclosed herein.

Although portions of the discussion herein may relate to an imager or an image sensor, embodiments of the invention are not limited in this regard; such imager or image sensor may include, for example, a detector, a sensor, a photodiode, a florescence device, an electrochemical sensing device, a magnetic field sensing device, a spectrophotometer, an image sensor, a Charge Coupled Device (CCD) camera or imager, a Complementary Metal Oxide Semiconductor (CMOS) camera or imager, a digital camera, a stills camera, a video camera, a light sensor; a device capable of detecting or sensing one or more colors, intensities, hues, brightness, contrast, and/or other parameters or characteristic; a device sensitive to one or more colors or able to detect one or more colors; a device capable of detecting one or more color changes; a device sensitive to color changes; or the like

A device, system and method in accordance with some embodiments of the invention may be used, for example, in conjunction with a device which may be inserted into a human body. However, the scope of the present invention is not limited in this regard. For example, some embodiments of the invention may be used in conjunction with a device which may be inserted into a non-human body or an animal body.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An autonomous in-vivo device comprising:
a suction chamber to store a sample;
a plunger movable into an inner portion of said suction chamber;
an imager to acquire an image in-vivo; and
a transmitter to transmit said image.

2. The autonomous in-vivo device of claim 1, comprising a blade to cut said sample from a lumen wall.

3. The autonomous in-vivo device of claim 1, comprising a coil to move said plunger towards said inner portion of said suction chamber.

4. The autonomous in-vivo device of claim 3, wherein said coil is activated by an electric current.

5. The autonomous in-vivo device of claim 1, comprising a latch to hold said plunger at said inner portion of said suction chamber.

6. The autonomous in-vivo device of claim 2, wherein said blade is activated by a movement of said plunger into said inner portion of said suction chamber.

7. The autonomous in-vivo device of claim 2, comprising a spring to snap shut said blade over said suction chamber upon movement of said plunger into said inner portion of said suction chamber.

8. The autonomous in-vivo device of claim 2, wherein said blade is held in place, prior to its activation, by said plunger.

9. The autonomous in-vivo device of claim 1, wherein at least a circumferential edge of said plunger includes a lubricant.

10. The autonomous in-vivo device of claim 1, wherein at least a portion of said plunger is surrounded by a sealant.

11. The autonomous in-vivo device of claim 1, wherein a movement of said plunger is triggered by an external command.

12. The autonomous in-vivo device of claim 1, comprising an in-vivo sensor to trigger a movement of said plunger.

13. The autonomous in-vivo device of claim 1, wherein a movement of said plunger is triggered based on a location of said in-vivo device.

14. The autonomous in-vivo device of claim 1, comprising a plurality of suction chambers to collect a plurality of samples, respectively.

15. The autonomous in-vivo device of claim 14, wherein said plurality of suction chambers are positioned at a plurality of sides of said in-vivo device, respectively.

16. The autonomous in-vivo device of claim 14, wherein a first of said suction chambers is to collect a first sample at a first time, and a second of said suction chambers is to collect a second sample at a second, different time.

17. The autonomous in-vivo device of claim 14, wherein at least two of said suction chambers are to collect said samples substantially simultaneously.

18. The autonomous in-vivo device of claim 14, comprising a rotatable wheel having at least two blades to cut said samples.

19. The autonomous in-vivo device of claim 1, comprising an in-vivo sensor to sense a property of said sample stored in said suction chamber.

20. The autonomous in-vivo device of claim 1, wherein said imager is to acquire an in-vivo image of a body lumen.

21. The autonomous in-vivo device of claim 1, wherein said imager is to acquire an image of said sample stored in said suction chamber.

22. The autonomous in-vivo device of claim 1, wherein said in-vivo device comprises a swallowable capsule.

23. A system comprising:
an in-vivo device including at least a suction chamber to store a sample, and a plunger movable into an inner portion of said suction chamber; and
a receiver to receive data transmitted from said in-vivo device.

24. The system of claim 23, wherein said in-vivo device further comprises a blade to cut said sample from a lumen wall.

25. The system of claim 23, wherein said in-vivo device comprises:
an in-vivo sensor to sense a property of said sample stored in said suction chamber; and
a transmitter to transmit the sensed data.

26. The system of claim 23, wherein said in-vivo device comprises:
an in-vivo imager to acquire an image of said sample stored in said suction chamber; and
a transmitter to transmit the image data.

27. The system of claim 23, wherein said in-vivo device comprises:
an in-vivo camera to acquire an image of a body lumen; and
a transmitter to transmit the image data.

28. A method comprising:
creating suction in a suction chamber of an in-vivo device;
drawing in a body tissue into said suction chamber using said suction;
cutting a sample of said tissue;
acquiring an image in-vivo; and
transmitting said image.

29. The method of claim 28, wherein acquiring comprises:
acquiring in-vivo an image of said sample.

30. The method of claim 28, wherein acquiring comprises:
acquiring in-vivo an image of a body lumen.

31. The method of claim 28, further comprising:
sensing in-vivo a property of said sample.

32. The method of claim 28, further comprising:
analyzing in-vivo said sample.
